(19) United States
(12) Patent Application Publication
(10) Pub. No.: US 2007/0160584 A1
(43) Pub. Date: Jul. 12, 2007

(54) Method of bone regeneration

(76) Inventors: Minoru Ueda, Nagoya-shi (JP); Yusuke Ando, Kurashiki-shi (JP); Takayuki Ohara, Nagareyama-shi (JP); Hideaki Kagami, Nagoya-shi (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 10/567,926

(22) PCT Filed: Aug. 10, 2004
(86) PCT No.: PCT/JP04/11740

§ 371 Date: Sep. 13, 2006

(30) Foreign Application Priority Data
Aug. 11, 2003 (JP) 2003-291078

(51) Int. Cl. A61K 35/32; C12N 5/08

(52) U.S. Cl. 424/93.7; 623/23.61; 435/366

(57) ABSTRACT

It is an object of the present invention to provide a method for effectively regenerating bone, and more specifically to provide a method for regenerating bone that is capable of treating patients suffering from bone defect or bone injury. The present invention provides a method for regenerating bone, which comprises culturing and/or transplanting mesenchymal cells in the coexistence of epithelial cells.

### TECHNICAL FIELD

The present invention relates to a method for regenerating bone. More specifically, the present invention relates to a method for regenerating bone by transplantation of mesenchymal cells in the coexistence of epithelial cells. The present invention also relates to a method for treating patients using bone regenerated by the above method.

### BACKGROUND ART

Bone fracture is a disorder, which may occur in people at any age. In many cases, it takes much time to heal such bone fracture. Since bone fracture affects the daily life of patients, it is very important to heal bone fracture as early as possible in terms of QOL. In particular, in the case of bone fracture of aged people, there is a high risk of becoming confined to their bed for a long period of time. Thus, the bone fracture of aged people causes social and economical problems.

Examples of bone defect may include alveolar ridge atrophy, bone defect generated as a result of the extirpation of tumor or bouton, and bone defect due to external injuries or congenital disease (cleft palate, etc.). Such bone detect has been treated via bone transplantation, bone distraction, or the use of artificial bone. However, such treatments have not necessarily brought on sufficient effects. In addition, problems on the donor side (burden or risk imposed on patients) have still remained. With regard to the treatment of bone fracture and bone defect, the use of osteogenesis promoting factors such as BMP, FGF, or TGF-β has been studied. However, since such peptide factors are rapidly metabolized in living bodies and become inactivated, or since it is difficult to maintain the optimal concentration, sufficient therapeutic effects cannot be obtained in many cases. Moreover, although medicaments for improving the stability of such factors have been studied, satisfactory products which can be applied in clinical sites, have not yet been obtained.

Furthermore, low molecular weight compounds exhibiting osteogenesis-promoting action, such as prostaglandins, benzylphosphonic acid derivatives, phenolsulfophthalein derivatives, or vitamin D derivatives, have also been studied. However, under the present circumstances, such low molecular weight compounds may have side effects, or may not have capacity sufficient for the clinical treatment of bone fracture or bone defect.

In recent years, in order to radically solve the aforementioned problems, a treatment using cells derived from homologous or autologous bone has been studied. Namely, a technique of transplanting into a bone fracture site or bone defect site osteoblasts playing a main role of osteogenesis or osteoblasts obtained by differentiation of bone marrow-derived undifferentiated mesenchymal stem cells together with a suitable carrier, has been attempted (Ohgushi et al., J. Biomed. Mat. Res. (48), 913-927, 1999). This technique is anticipated to be an effective technique causing few side effects. However, this is a technique that is still insufficient in terms of the amount of bone formed, treatment period, etc.

As stated above, when bone is formed using cells, blast cells that form tissues or the precursor cells thereof, or mesenchymal cells such as stem cells alone, have been generally used. Thus, no techniques of allowing epithelial cells to coexist with the aforementioned cells so as to significantly promote osteogenesis have been known.

### DISCLOSURE OF THE INVENTION

It is an object of the present invention to solve the aforementioned problems of the prior art techniques. That is to say, it is an object of the present invention to provide a method for effectively regenerating bone, and more specifically to provide a method for regenerating bone that is capable of treating patients suffering from bone defect or bone injury. Moreover, it is another object of the present invention to provide a method for treating patients suffering from bone defect or bone injury using the regenerated bone.

As a result of intensive studies directed towards achieving the aforementioned objects, the present inventors have found that induction of differentiation of mesenchymal cells can be promoted by culturing and/or transplanting the mesenchymal cells in the coexistence of epithelial cells, so that bone regeneration can be promoted, thereby completing the present invention.

Thus, the present invention provides a method for regenerating bone, which comprises culturing mesenchymal cells in the coexistence of epithelial cells. Preferably, the mesenchymal cells are cultured on a carrier in the coexistence of epithelial cells.

In another aspect, the present invention provides a method for regenerating bone, which comprises transplanting mesenchymal cells into an animal in the coexistence of epithelial cells, and regenerating bone in the transplanted animal. Preferably, mesenchymal cells are transplanted into an animal together with a carrier in the coexistence of epithelial cells, and bone is regenerated in the body of the transplanted animal.

Preferred examples of epithelial cells used herein may include inner enamel epithelial cells, outer enamel epithelial cells, enamel pulp cells, intermediate layer cells, ameloblasts, Malassez's epithelial rest cells, oral mucous membrane epidermic cells, epidermic cells, epidermal cells, and their precursor cells. Preferred examples of mesenchymal cells used herein may include odontoblasts, pulp cells, dental papilla cells, tooth sac cells, cementoblasts, osteoblasts, their precursor cells, and mesenchymal stem cells. Bone to be regenerated is preferably jawbone or alveolar bone.

In another aspect, the present invention provides bone regenerated by the aforementioned method of the present invention. In a further aspect, the present invention provides a therapeutic method, which comprises transplanting the bone regenerated by the aforementioned method of the present invention into a patient suffering from bone defect or bone injury. In a further aspect, the present invention provides a composition for bone regeneration, which comprises: (1) epithelial cells selected from among inner enamel epithelial cells, outer enamel epithelial cells, enamel pulp cells, intermediate layer cells, ameloblasts, Malassez's epithelial rest cells, oral mucous membrane epidermic cells, epidermic cells, epidermal cells, and their precursor cells; (2) mesenchymal cells selected from among odontoblasts, pulp cells, dental papilla cells, tooth sac cells, cementoblasts, osteoblasts, their precursor cells, and mesenchymal stem cells; and (3) a carrier.

### BRIEF DESCRIPTION OF THE DRAWINGS

FIG. 1 shows a transplant obtained by inoculating tooth germ mesenchymal cells alone on a carrier, transplanting the obtained product into an animal, and extirpating it 11 weeks after the transplantation.

FIG. 2 shows a histology of tissues (stained with hematoxylin and eosin) of a transplant, which was obtained by inoculating tooth germ mesenchymal cells alone on a carrier, transplanting the obtained product into an animal, and extirpating it 11 weeks after the transplantation.

FIG. 3 shows a histology of tissues (stained with hematoxylin and eosin) of a transplant, which was obtained by inoculating the cultured tooth germ mesenchymal cells alone on a carrier, transplanting the obtained product into an animal, and extirpating it 4 weeks after the transplantation.

FIG. 4 shows a transplant obtained by inoculating a mixture of tooth germ epithelial cells and tooth germ mesenchymal cells on a carrier, transplanting the obtained product into an animal, and extirpating it 4 weeks after the transplantation.

FIG. 5 shows a histology of tissues (stained with hematoxylin and eosin) of a transplant, which was obtained by inoculating a mixture of tooth germ epithelial cells and tooth germ mesenchymal cells on a carrier, transplanting the obtained product into an animal, and extirpating it 4 weeks after the transplantation.

FIG. 6 shows a transplant obtained by inoculating tooth germ epithelial cells and tooth germ mesenchymal cells on a carrier separately, transplanting the obtained product into an animal, and extirpating it 4 weeks after the transplantation.

FIG. 7 shows a histology of tissues (stained with hematoxylin and eosin) of a transplant, which was obtained by inoculating tooth germ epithelial cells and tooth germ mesenchymal cells on a carrier separately, transplanting the obtained product into an animal, and extirpating it 4 weeks after the transplantation.

FIG. 8 shows a transplant obtained by inoculating tooth germ mesenchymal cells on a carrier, wrapping the obtained product with an oral mucous membrane epidermic cell sheet, transplanting it into an animal, and extirpating it 4 weeks after the transplantation.

FIG. 9 shows a histology of tissues (stained with hematoxylin and eosin) of a transplant, which was obtained by inoculating tooth germ mesenchymal cells on a carrier, wrapping the obtained product with an oral mucous membrane epidermic cell sheet, transplanting it into an animal, and extirpating it 4 weeks after the transplantation.

FIG. 10 shows a transplant obtained by inoculating a mixture of the cultured tooth germ mesenchymal cells and epidermal cells on a carrier, transplanting the obtained product into an animal, and extirpating it 4 weeks after the transplantation.

FIG. 11 shows a histology of tissues (stained with hematoxylin and eosin) of a transplant, which was obtained by inoculating a mixture of the cultured tooth germ mesenchymal cells and epidermal cells on a carrier, transplanting the obtained product into an animal, and extirpating it 4 weeks after the transplantation.

### BEST MODE FOR CARRYING OUT THE INVENTION

The embodiments of the present invention will be described in detail below. The method for regenerating bone according to the present invention is characterized in that mesenchymal cells are cultured and/or are transplanted into a transplantation animal in the coexistence of epithelial cells, so as to regenerate bone.

The type of an epithelial cell used in the present invention is not particularly limited, as long as it is an epithelial cell. Preferred examples of such epithelial cells may include inner enamel epithelial cells, outer enamel epithelial cells, enamel pulp cells, intermediate layer cells, ameloblasts, Malassez's epithelial rest cells, oral mucous membrane epidermic cells, epidermic cells, epidermal cells, and their precursor cells. Such cells may be cultured or transplanted after separation, in the form of single cells consisting of one type of epithelial cell. Otherwise, they may also be cultured or transplanted after separation, in the form of a cell mixture consisting of two or more types of epithelial cells.

On the other hand, the type of a mesenchymal cell used in the present invention is not particularly limited, as long as it is a mesenchymal cell. Preferred examples of such mesenchymal cells may include odontoblasts, pulp cells, dental papilla cells, tooth sac cells, cementoblasts, osteoblasts, their precursor cells, and mesenchymal stem cells. Such cells may be cultured or transplanted after separation, in the form of single cells consisting of one type of mesenchymal cell. Otherwise, they may also be cultured or transplanted after separation, in the form of a cell mixture consisting of two or more types of mesenchymal cells.

Epithelial cells can be collected from tooth germ, periodontium (Malassez's epithelial rest), oral mucous membrane, junctional epithelium, skin, or the like, of mammals (for example, a human, a swine, etc.) according to a known method. For example, in the case of epithelial cells such as inner enamel epithelial cells, outer enamel epithelial cells, enamel pulp cells, intermediate layer cells, or ameloblasts, such cells can be collected from the lower jawbone of a mammal (for example, a human, a swine, etc.). An impacted tooth is aseptically excised, and it is then conserved in a suitable medium such as a Hanks balanced salt solution (HBSS). A calcified portion is removed from the tooth, and the residual tissues are fragmented using a knife. The fragmented tissues are then washed with an HBSS solution or the like. Subsequently, the tissues are preferably subjected to an enzyme treatment with collagenase and dispase. After completion of such an enzyme treatment, cells can be recovered by pipetting and centrifugation. When the obtained cells are cultured using MCDB153 (Kyokuto Co.) as a medium, mesenchymal cells contained in the tooth germ are lost, so as to obtain only epithelial cells.

In addition, in the case of oral mucous membrane epidermic cells, oral mucous membrane collected from a human is treated with dispase. Thereafter, the epithelial portion is peeled, followed by a treatment with trypsin, thereby obtaining the epidermic cells.

Mesenchymal cells can be collected from tooth germ, dental pulp, alveolar bone, bone marrow, or the like, of mammals (for example, a human, a swine, etc.) according to a known method. For example, mesenchymal cells contained in tooth germ can be collected from the lower jawbone of a mammal (for example, a human, a swine, etc.). An impacted tooth is aseptically excised, and it is then conserved in a suitable medium such as a PBS solution or an HBSS solution. A calcified portion is removed from the tooth, and the residual tissues are fragmented using a knife. The fragmented tissues are then washed with a PBS solution, an HBSS solution, or the like. Subsequently, the tissues are preferably subjected to an enzyme treatment with collagenase and dispase. After completion of such an enzyme treatment, cells can be recovered by pipetting and centrifugation. When the obtained cells are sub-cultured using a medium formed by adding 10% fetal bovine serum and 1% antibiotics to Dulbecco's Modified Eagle Medium, epithelial cells contained in the tooth germ are lost, so as to obtain only mesenchymal cells.

Dental pulp can be extirpated from a tooth according to the method described in About I. et al., Experimental Cell Research. 258. 33-41, 2000. Aseptically collected dental pulp is transferred to a petri dish, and it is then cultured in a medium, thereby obtaining mesenchymal cells.

Moreover, bone marrow is collected from ilium or the like via bone marrow biopsy according to a known method, and it is then cultured, thereby obtaining mesenchymal stem cells.

The bone regenerated by the method of the present invention is transplanted to a patient (that is, a patient who suffers from bone defect or bone injury), and thus it is used for the treatment of such a patient. In this case, from the viewpoint of biocompatibility associated with transplantation, cells used in regeneration are preferably the patient's own cells. However, it is also possible to use homologous (allogeneic) cells. When cells constituting tooth germ or cells differentiating into tooth germ are used, such cells can also be collected from wisdom teeth.

It has been known that a tooth is formed in 5 stages ranging from generation to maturation. The first stage is called the Initiation stage, when epithelial tissues and mesenchymal tissues are induced to the basement membrane. The second stage is called the Bud stage, when an enamel organ is generated. The third stage is called the Cap stage, when dental papilla is formed and tooth germ is then formed. The fourth stage is called the Bell stage, when both differentiation of the tooth germ into cells forming dental enamel and differentiation of the dental papilla into cells forming dentin and dental pulp are initiated. The fifth stage is called the Maturation stage, when cells are differentiated into tissues constituting the tooth, such as dental enamel, dentin, and dental pulp. In the present invention, cells in a preferred stage selected from the aforementioned stages can be collected and used. In a case where no tooth germ exists, dental pulp is excised from a tooth root, and cells can be then separated and collected therefrom.

Cells can be cultured, using a common serum-containing medium that is used in the culture of animal cells, under common conditions for culturing animal cells (for example, at a temperature between room temperature and 37° C., in a 5% to 10% CO₂ incubator, etc.). In addition, when epithelial cells are cultured, it is possible to culture them using a serum free medium, or it is also possible to culture them in the coexistence of feeder cells such as fibroblasts.

In the present invention, cells may be cultured on a carrier, or may be cultured with no carriers. However, cells are preferably cultured on a carrier. The use of a carrier is useful for forming bone from the cells. It is preferable to use a carrier, which endures a period of time necessary for formation of bone, and which is then rapidly absorbed into a body. That is to say, it is preferable to use a carrier, which has a suitable speed and properties of being absorbed into a living body such as the subcutis, the greater omentum attached to the stomach, or the jawbone, and which is produced from a material having high affinity to the cells.

The material of the carrier is not particularly limited, as long as it satisfies the aforementioned properties. Examples of such a material may include: synthetic polymer materials such as polyglycolic acid (PGA), poly(DL-lactide-co-glycolide) (PLGA), polylactic acid (PLLA), or polycaprolactone; protein materials such as collagen, gelatin, or fibrin; and natural materials such as hyaluronic acid or a salt thereof, alginic acid or a salt thereof, dentin, or coral. Also, inorganic materials such as tricalcium phosphate (β-TCP) may be used.

PGA is commercially available from Albany International Research Co. and other companies. PLGA is commercially available from Sigma. In the case of PGA, since this compound is rapidly absorbed, it is also possible to coat the surface thereof with poly(DL-lactide) (PLLA), so as to retard the absorption period. Moreover, when synthetic materials such as PGA, PLLA, PLGA or polycaprolactone are used, in order to enhance the adhesiveness and proliferative properties of the cells, the surfaces of these compounds may be coated with a collagen solution, a fibronectin solution, or the like, and then used.

Examples of a possible form of the aforementioned carrier may include a mesh form, a sponge form, a gel form, and a non-woven form.

A carrier processed into a form, which facilitates transplantation of the cells, is preferable. Such a carrier preferably has a platy or spherical porous form, or hollow form, one end of which is open, so that blood can easily be introduced from surrounding portions.

It is preferable to produce a carrier with a form that is suitable for purpose. Thus, a form of interest is produced from resin, and a mold is then obtained using an impression material. Thereafter, the mold of resin is taken out, and a synthetic material constituting a carrier is poured therein, so as to replicate the form of interest.

In the method of the present invention, it may be possible that epithelial cells and mesenchymal cells be cultured, and that the cultured epithelial and mesenchymal cells be then transplanted into a transplantation animal, so as to regenerate bone in the body of the transplanted animal. Alternatively, it may also be possible that the above epithelial cells and mesenchymal cells be directly transplanted into the bone of a patient, or the like. Preferably, a carrier used in the culture of cells is also transplanted into the body of the transplantation animal, together with the cells.

The type of a transplanted animal is not particularly limited, but it is preferably a mammal. Examples of a mammal used herein may include rodents such as a rat (e.g. a hairless rat), rabbit, or mouse. As a site into which cells are transplanted, a site to which factors necessary for formation of bone can easily be supplied is preferable. Specifically, a site having a high blood flow, such as the greater omentum attached to the stomach in the abdominal cavity, is particularly preferable. By transplanting cells into such a site, the growth of the cells can be promoted, and formation of bone can be accelerated.

Bone regenerated by the above-described method for regenerating bone according to the present invention (which may be either bone obtained by culturing cells, or bone obtained by transplanting the above bone into a transplantation animal and allowing it to further regenerate in the body of the transplantation animal) is transplanted to a patient who suffers from bone defect or bone injury, so as to treat the patient. That is to say, a method for treating a patient using bone obtained by the method for regenerating bone according to the present invention is also included in the scope of the present invention. Even after bone has been transplanted to a patient, the bone may be allowed to continuously grow, so as to further form bone.

The present invention will be further specifically described in the following examples. However, the examples are not intended to limit the scope of the present invention.

### EXAMPLES

### Comparative Example 1

### Transplantation of Tooth Germ Mesenchymal Cells Alone

A lower jawbone was collected from a fresh swine with an age of 6 months old. The collected bone was conserved in a refrigerator at 4° C. until it was used in an experiment. During transportation, the bone was conserved on ice. An impacted tooth was aseptically excised, and it was then conserved in a 10% antibiotics-containing phosphate buffered saline (PBS).

The thus excised impacted tooth was subjected to an enzyme treatment for 120 minutes using an enzyme solution prepared by dissolving 200 PU/ml dispase in Dulbecco's Modified Eagle Medium (DMEM). Thereafter, the impacted tooth was separated into epithelial cells-containing tissues and mesenchymal cells-containing tissues, using a knife. A calcified portion was removed from each type of the thus separated tissues. Thereafter, using a knife, the residual tissues were then fragmented into fragments of a size of approximately 2 mm, and the fragments were then washed with a PBS solution 5 times.

Using an enzyme solution prepared by dissolving 2 mg/ml collagenase in DMEM medium, the washed tissues that contained mesenchymal cells alone were subjected to an enzyme treatment for 50 minutes. The obtained tissues were subjected to pipetting using a 25-ml pipette for 10 minutes. 25 ml of a supernatant was then centrifuged (1,500 rpm, 5 minutes), so as to recover cells. The obtained cells were washed with 10% serum-containing DMEM medium 5 times, and they were then centrifuged, so as to recover cells.

The recovered mesenchymal cells were added to DMEM medium, so as to prepare a cell suspension having a concentration of 1.5×10⁷ cells/100 μl. The cell suspension was inoculated on a PGA mesh carrier (volume density: 50% to 60%; thickness: 2 mm; manufactured by Albany International Research, MA, U.S.A.), followed by a static culture at 37° C. in 5% CO₂ for 24 hours.

As a transplantation animal, a nude rat F344 was used. The abdominal skin of such a nude rat was incised, and its greater omentum was pulled out. The carrier, on which the mesenchymal cells had been inoculated, was wrapped with the above greater omentum, and it was then sutured. Thereafter, the muscle coat and the skin were sutured.

11 weeks after the transplantation, a sample was collected. The extirpated sample was fixed with a 10% formalin solution, and it was then embedded in paraffin according to a common method, so as to produce a continuous tissue section. Thereafter, the section was stained with hematoxylin and eosin, and thus it was observed in a histological manner.

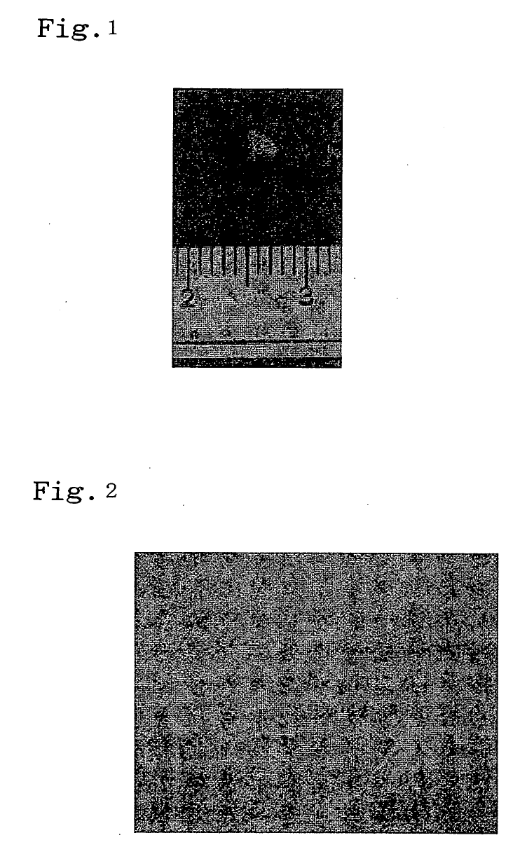

The transplant that had been extirpated 11 weeks after the transplantation was a tissue having a diameter of approximately 3.5 mm (FIG. 1). Thereafter, the tissue stained with hematoxylin and eosin was observed. As a result, it was found that almost no hard tissues were formed (FIG. 2).

### Comparative Example 2

### Transplantation of Cultured Tooth Germ Mesenchymal Cells Alone

A lower jawbone was collected from a fresh swine with an age of 6 months old. The collected bone was conserved in a refrigerator at 4° C. until it was used in an experiment. During transportation, the bone was conserved on ice. An impacted tooth was aseptically excised, and it was then conserved in a 10% antibiotics-containing PBS solution.

The thus excised impacted tooth was subjected to an enzyme treatment for 120 minutes using an enzyme solution prepared by dissolving 200 PU/ml dispase in DMEM medium. Thereafter, the impacted tooth was separated into epithelial cells-containing tissues and mesenchymal cells-containing tissues, using a knife. A calcified portion was removed from each type of the thus separated tissues. Thereafter, using a knife, the residual tissues were then fragmented into fragments of a size of approximately 2 mm, and the fragments were then washed with a PBS solution 5 times.

Using an enzyme solution prepared by dissolving 2 mg/ml collagenase in DMEM medium, the washed tissues that contained mesenchymal cells alone were subjected to an enzyme treatment for 50 minutes. The obtained tissues were subjected to pipetting using a 25-ml pipette for 10 minutes. 25 ml of a supernatant was then centrifuged (1,500 rpm, 5 minutes), so as to recover cells. The obtained cells were washed with 10% serum-containing DMEM medium 5 times, and they were then centrifuged, so as to recover cells.

The recovered cells were cultured in DMEM medium at 37° C. in 5% CO₂, so as to acquire necessary number of cells. The thus obtained cells were removed from a flask used for cell culture, using trypsin-EDTA, and they were then inoculated on a PGA mesh carrier, followed by a static culture at 37° C. in 5% CO₂ for 24 hours.

As a transplantation animal, a KSN/slc nude mouse was used. The epidermis of the nude mouse was incised, and the muscle coat and the epidermis were then peeled, so as to make a space. Thereafter, the PGA mesh carrier, on which the mesenchymal cells had been inoculated, was transplanted into the empty space.

4 weeks after the transplantation, a sample was collected. The extirpated sample was fixed with a 10% formalin solution, and it was then embedded in paraffin according to a common method, so as to produce a continuous tissue section. Thereafter, the section was stained with hematoxylin and eosin, and thus it was observed in a histological manner.

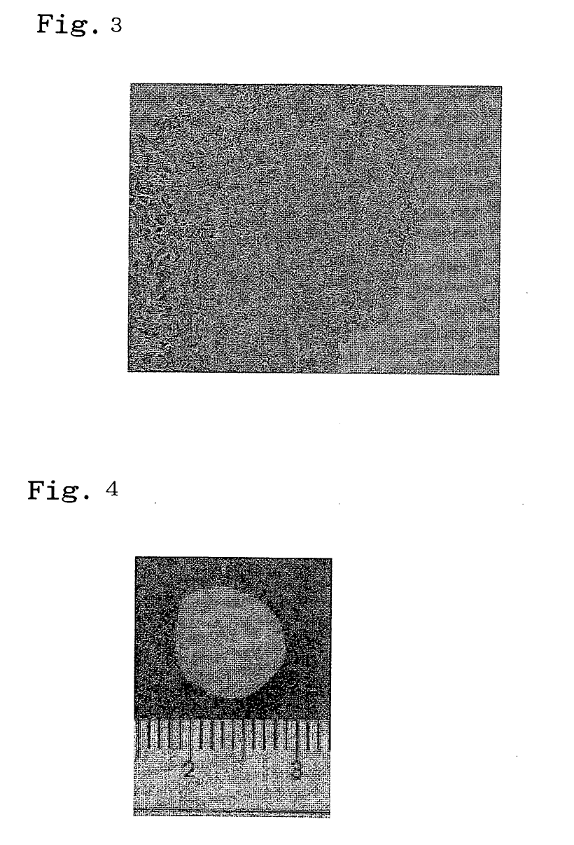

The transplant that had been extirpated 4 weeks after the transplantation was stained with hematoxylin and eosin, and the thus obtained tissue was then observed. As a result, it was found that almost no hard tissues were formed (FIG. 3).

### Example 1

### Transplantation of Mixture of Tooth Germ Epithelial Cells and Tooth Germ Mesenchymal Cells

A lower jawbone was collected from a fresh swine with an age of 6 months old. The collected bone was conserved in a refrigerator at 4° C. until it was used in an experiment. During transportation, the bone was conserved on ice. An impacted tooth was aseptically excised, and it was then conserved in a 10% antibiotics-containing PBS solution. A calcified portion was removed from the tooth germ, and using a knife, the residual tissues were then fragmented into fragments of a size of approximately 2 mm. The fragments were then washed with a PBS solution 5 times.

Using an enzyme solution prepared by dissolving 2 mg/ml collagenase in DMEM medium, the washed tissues were subjected to an enzyme treatment for 50 minutes. The obtained tissues were subjected to pipetting using a 25-ml pipette for 10 minutes. 25 ml of a supernatant was then centrifuged (1,500 rpm, 5 minutes), so as to recover cells. The obtained cells were washed with 10% serum-containing DMEM medium 5 times, and they were then centrifuged, so as to recover mixed cells of tooth germ epithelial cells and tooth germ mesenchymal cells.

The recovered mixed cells were added to DMEM medium, so as to prepare a cell suspension having a concentration of 1.5×10⁷ cells/100 μl. The cell suspension was inoculated on a PGA mesh carrier. The carrier, on which the cells had been inoculated, was subjected to a static culture for 24 hours. As a medium for culturing the cells, a medium formed by adding 10% fetal bovine serum and antibiotics to DMEM was used. In addition, the cells were cultured at 37° C. in 5% CO₂.

As a transplantation animal, a KSN/slc nude mouse was used. The epidermis of the nude mouse was incised, and the muscle coat and the epidermis were then peeled, so as to make a space. Thereafter, the PGA mesh, on which the cells had been inoculated, was transplanted into the empty space.

4 weeks after the transplantation, a sample was collected. The extirpated sample was fixed with a 10% formalin solution, and it was then embedded in paraffin according to a common method, so as to produce a continuous tissue section. Thereafter, the section was stained with hematoxylin and eosin, and thus it was observed in a histological manner.

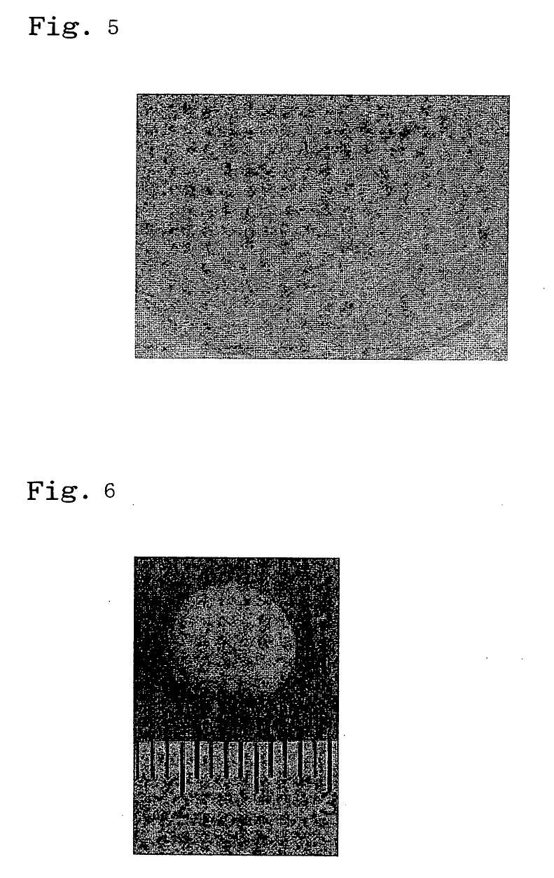

The transplant that had been extirpated 4 weeks after the transplantation was a hard tissue with a diameter of approximately 10 mm (FIG. 4). It was confirmed that this hard tissue was significantly greater than the tissue obtained using mesenchymal cells alone in Comparative example 1 (which was hardly calcified). Moreover, the tissue stained with hematoxylin and eosin was observed. As a result, it was found that an osteoid tissue was formed in the tissue (FIG. 5). From the results of Comparative examples 1 and 2, no formation of hard tissues was observed. In addition, to date, such significant formation of osteoid tissues has never been observed in such a short time. Accordingly, it is considered that the growth of osteoid tissues was promoted by addition of epithelial cells.

### Example 2

### Transplantation of Tooth Germ Epithelial Cells and Tooth Germ Mesenchymal Cells, which have been Inoculated Separately

A lower jawbone was collected from a fresh swine with an age of 6 months old. The collected bone was conserved in a refrigerator at 4° C. until it was used in an experiment. During transportation, the bone was conserved on ice. An impacted tooth was aseptically excised, and it was then conserved in a 10% antibiotics-containing PBS solution.

The thus excised impacted tooth was subjected to an enzyme treatment for 120 minutes using an enzyme solution prepared by dissolving 200 PU/ml dispase in DMEM medium. Thereafter, the impacted tooth was separated into epithelial cells-containing tissues and mesenchymal cells-containing tissues, using a knife. A calcified portion was removed from each type of the thus separated tissues, and using a knife, the residual tissues were then fragmented into fragments of a size of approximately 2 mm. The fragments were then washed with a PBS solution 5 times.

Using an enzyme solution prepared by dissolving 2 mg/ml collagenase in DMEM medium, each type of the washed tissues were subjected to an enzyme treatment for 50 minutes. The obtained tissues were subjected to pipetting using a 25-ml pipette for 10 minutes. 25 ml of a supernatant was then centrifuged (1,500 rpm, 5 minutes), so as to recover cells. The obtained cells were washed with 10% serum-containing DMEM medium 5 times, and they were then centrifuged, so as to recover tooth germ epithelial cells and tooth germ mesenchymal cells, separately.

The recovered mesenchymal cells were added to DMEM medium, so as to prepare a cell suspension having a concentration of 1.5×10⁷ cells/100 μl. The cell suspension was inoculated on a PGA mesh carrier.

On the other hand, the recovered epithelial cells was added to a solution produced with type I collagen (a solution that is gelatinized at 37° C.), so as to prepare a cell suspension having a concentration of 1.5×10⁷ cells/100 μl.

The PGA mesh carrier, on which the cells had been inoculated, was subjected to a static culture for 1 hour. Thereafter, the resultant carrier was coated with the collagen solution in which epithelial cells had been suspended, and it was then subjected to a static culture for 1 hour.

Thereafter, a sufficient amount of DMEM medium was added to the culture product, and the obtained mixture was then subjected to a static culture for 24 hours. The cell culture was carried out at 37° C. in 5% CO₂.

As a transplantation animal, a KSN/slc nude mouse was used. The epidermis of the nude mouse was incised, and the muscle coat and the epidermis were then peeled, so as to make a space. Thereafter, the PGA mesh carrier coated with the collagen gel containing the cells was transplanted into the empty space.

4 weeks after the transplantation, a sample was collected. The extirpated sample was fixed with a 10% formalin solution, and it was then embedded in paraffin according to a common method, so as to produce a continuous tissue section. Thereafter, the section was stained with hematoxylin and eosin, and thus it was observed in a histological manner.

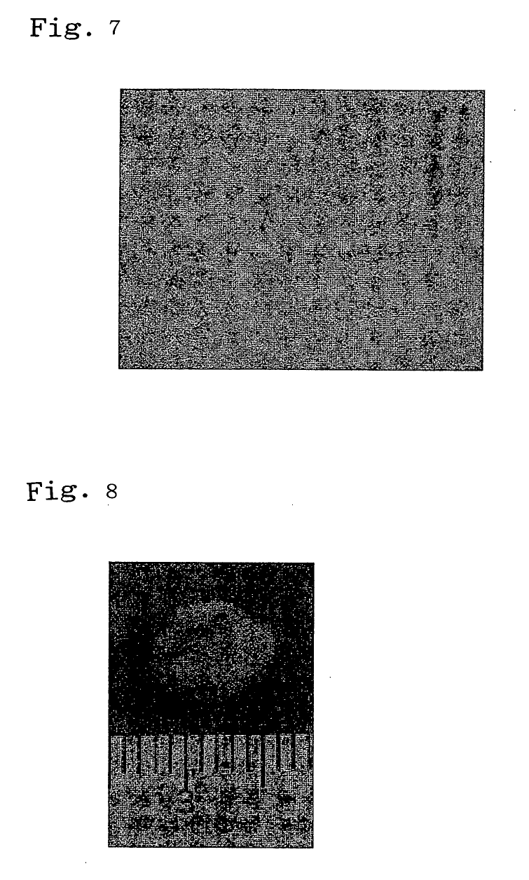

The transplant that had been extirpated 4 weeks after the transplantation was a hard tissue of a size of approximately 9 mm (FIG. 6). It was confirmed that this hard tissue was significantly greater than the tissue obtained using mesenchymal cells alone in Comparative example 1 (which was hardly calcified). Moreover, the tissue stained with hematoxylin and eosin was observed. As a result, it was found that an osteoid tissue was formed in the tissue (FIG. 7). From the results of Comparative examples 1 and 2, no formation of hard tissues was observed. In addition, to date, such significant formation of osteoid tissues has never been observed in such a short time. Accordingly, it is considered that the growth of osteoid tissues was promoted by addition of epithelial cells.

### Example 3

### Transplantation of Tooth Germ Mesenchymal Cells Wrapped with Oral Mucous Membrane Epidermic Cell Sheet

A lower jawbone was collected from a fresh swine with an age of 6 months old. The collected bone was conserved in a refrigerator at 4° C. until it was used in an experiment. During transportation, the bone was conserved on ice. An impacted tooth was aseptically excised, and it was then conserved in a 10% antibiotics-containing PBS solution.

The thus excised impacted tooth was subjected to an enzyme treatment for 120 minutes using an enzyme solution prepared by dissolving 200 PU/ml dispase in DMEM medium. Thereafter, the impacted tooth was separated into epithelial cells-containing tissues and mesenchymal cells-containing tissues, using a knife. A calcified portion was removed from each type of the thus separated tissues, and using a knife, the residual tissues were then fragmented into fragments of a size of approximately 2 mm. The fragments were then washed with a PBS solution 5 times.

Using an enzyme solution prepared by dissolving 2 mg/ml collagenase in DMEM medium, the washed tissues that contained mesenchymal cells alone were subjected to an enzyme treatment for 50 minutes. The obtained tissues were subjected to pipetting using a 25-ml pipette for 10 minutes. 25 ml of a supernatant was then centrifuged (1,500 rpm, 5 minutes), so as to recover cells. The obtained cells were washed with 10% serum-containing DMEM medium 5 times, and they were then centrifuged, so as to recover the cells.

The recovered tooth germ mesenchymal cells were added to DMEM medium, so as to prepare a cell suspension having a concentration of 1.5×10⁷ cells/100 μl. The cell suspension was inoculated on a PGA mesh carrier, followed by a static culture for 1 hour at 37° C. in 5% CO₂.

The PGA mesh, on which the tooth germ mesenchymal cells had been inoculated, was wrapped with an oral mucous membrane cell sheet obtained by culturing human oral mucous membrane cells according to a common method, followed by a static culture for 24 hours. The culturing of the cells was carried out at 37° C. in 5% CO₂.

As a transplantation animal, a KSN/slc nude mouse was used. The epidermis of the nude mouse was incised, and the muscle coat and the epidermis were then peeled, so as to make a space. Thereafter, a carrier formed by wrapping the PGA mesh with the oral mucous membrane cell sheet was transplanted into the empty space.

4 weeks after the transplantation, a sample was collected. The extirpated sample was fixed with a 10% formalin solution, and it was then embedded in paraffin according to a common method, so as to produce a continuous tissue section. Thereafter, the section was stained with hematoxylin and eosin, and thus it was observed in a histological manner.

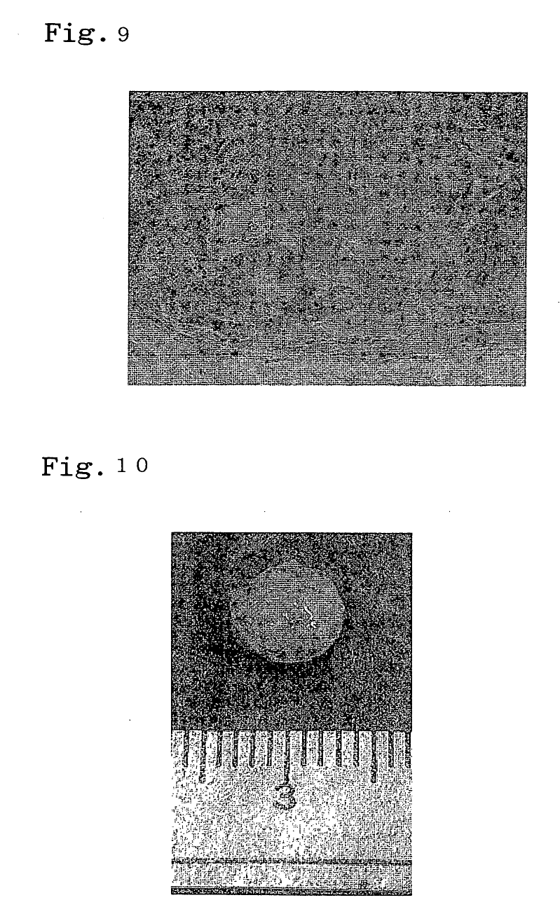

The transplant that had been extirpated 4 weeks after the transplantation was a hard tissue of a size of approximately 8 mm (FIG. 8). It was confirmed that this hard tissue was significantly greater than the tissue obtained using mesenchymal cells alone in Comparative example 1 (which was hardly calcified). Moreover, the tissue stained with hematoxylin and eosin was observed. As a result, it was found that an osteoid tissue was formed in the tissue (FIG. 9). From the results of Comparative examples 1 and 2, no formation of hard tissues was observed. In addition, to date, such significant formation of osteoid tissues has never been observed in such a short time. Accordingly, it is considered that the growth of osteoid tissues was promoted by addition of epithelial cells.

### Example 4

### Transplantation of Mixture of Cultured Tooth Germ Mesenchymal Cells and Epidermal Cells

A lower jawbone was collected from a fresh swine with an age of 6 months old. The collected bone was conserved in a refrigerator at 4° C. until it was used in an experiment. During transportation, the bone was conserved on ice. An impacted tooth was aseptically excised, and it was then conserved in a 10% antibiotics-containing PBS solution.

The thus excised impacted tooth was subjected to an enzyme treatment for 120 minutes using an enzyme solution prepared by dissolving 200 PU/ml dispase in DMEM medium. Thereafter, the impacted tooth was separated into epithelial cells-containing tissues and mesenchymal cells-containing tissues, using a knife. A calcified portion was removed from each type of the thus separated tissues, and using a knife, the residual tissues were then fragmented into fragments of a size of approximately 2 mm. The fragments were then washed with a PBS solution 5 times.

Using an enzyme solution prepared by dissolving 2 mg/ml collagenase in DMEM medium, the washed tissues that contained mesenchymal cells alone were subjected to an enzyme treatment for 50 minutes. The obtained tissues were subjected to pipetting using a 25-ml pipette for 10 minutes. 25 ml of a supernatant was then centrifuged (1,500 rpm, 5 minutes), so as to recover cells. The obtained cells were washed with 10% serum-containing DMEM medium 5 times, and they were then centrifuged, so as to recover the cells.

The recovered cells were cultured in DMEM medium at 37° C. in 5% CO₂. The cultured cells were removed from a flask used for cell culture, using trypsin-EDTA, thereby obtaining 5×10⁶ cells.

On the other hand, epidermal cells were collected from a Fisher rat according to a common method, and they were then cultured, so as to obtain epidermal cell sheets (two sheets for 75-cm² culture flasks). The obtained cell sheets were removed using trypsin-EDTA, and pipetting was then performed thereon, so as to obtain a cell suspension.

The aforementioned tooth germ mesenchymal cells are mixed with the epidermal cells, followed by suspension. Thereafter, the obtained suspension was inoculated on a PGA mesh carrier. Thereafter, a static culture was carried out at 37° C. in 5% CO₂.

As a transplantation animal, a KSN/slc nude mouse was used. The epidermis of the nude mouse was incised, and the muscle coat and the epidermis were then peeled, so as to make a space. Thereafter, the PGA mesh, on which the cells had been inoculated, was transplanted into the empty space.

4 weeks after the transplantation, a sample was collected. The extirpated sample was fixed with a 10% formalin solution, and it was then embedded in paraffin according to a common method, so as to produce a continuous tissue section. Thereafter, the section was stained with hematoxylin and eosin, and thus it was observed in a histological manner.

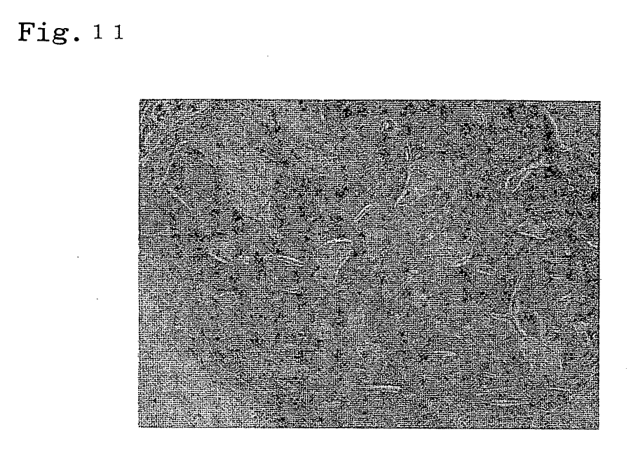

The transplant that had been extirpated 4 weeks after the transplantation was a hard tissue of a size of approximately 7 mm (FIG. 10). It was confirmed that this hard tissue was significantly greater than the tissue obtained using tooth germ mesenchymal cells alone in Comparative example 2 (which was hardly calcified). Moreover, the tissue stained with hematoxylin and eosin was observed. As a result, it was found that an osteoid tissue was formed in the tissue (FIG. 11). From the results of Comparative examples 1 and 2, no formation of hard tissues was observed. In addition, to date, such significant formation of osteoid tissues has never been observed in such a short time. Accordingly, it is considered that the growth of osteoid tissues was promoted by addition of epithelial cells.

### INDUSTRIAL APPLICABILITY

According to the method of the present invention, bone can be effectively regenerated.

1. A method for regenerating bone, which comprises culturing mesenchymal cells in the coexistence of epithelial cells.

2. The method for regenerating bone according to claim 1, which comprises culturing the mesenchymal cells on a carrier in the coexistence of epithelial cells.

3. A method for regenerating bone, which comprises transplanting mesenchymal cells into an animal in the coexistence of epithelial cells, and regenerating bone in the transplanted animal.

4. The method for regenerating bone according to claim 3, which comprises transplanting mesenchymal cells into an animal together with a carrier in the coexistence of epithelial cells, and regenerating bone in the body of the transplanted animal.

5. The method for regenerating bone according to claim 1, wherein the epithelial cells to be used are inner enamel epithelial cells, outer enamel epithelial cells, enamel pulp cells, intermediate layer cells, ameloblasts, Malassez's epithelial rest cells, oral mucous membrane epidermic cells, epidermic cells, epidermal cells, or their precursor cells, and the mesenchymal cells to be used are odontoblasts, pulp cells, dental papilla cells, tooth sac cells, cementoblasts, osteoblasts, or their precursor cells, or mesenchymal stem cells.

6. The method for regenerating bone according to claim 1, wherein the bone to be regenerated is jawbone or alveolar bone.

7. A bone regenerated by the method of claim 1.

8. A therapeutic method, which comprises transplanting the bone regenerated by the method of claim 1 into a patient suffering from bone defect or bone injury.

9. A composition for bone regeneration, which comprises:
(1) epithelial cells selected from among inner enamel epithelial cells, outer enamel epithelial cells, enamel pulp cells, intermediate layer cells, ameloblasts, Malassez's epithelial rest cells, oral mucous membrane epidermic cells, epidermic cells, epidermal cells, and their precursor cells;
(2) mesenchymal cells selected from among odontoblasts, pulp cells, dental papilla cells, tooth sac cells, cementoblasts, osteoblasts, or their precursor cells, or mesenchymal stem cells; and
(3) a carrier.
